(19) United States
(12) Patent Application Publication
(10) Pub. No.: US 2023/0389790 A1
(43) Pub. Date: Dec. 7, 2023

(54) METHOD AND SYSTEM FOR CONDUCTING A VISION ASSESSMENT TEST USING AUTHORED TEST PROFILES

(71) Applicants: OCCUTRACK MEDICAL SOLUTIONS PTE. LTD., Singapore (SG); TAN TOCK SENG HOSPITAL PTE LTD, Singapore (SG)

(72) Inventors: Kei Fong Mark WONG, Singapore (SG); Chee Mun Eric LOH, Singapore (SG); Augustinus LAUDE, Valley Park, (SG); TOCK HAN LIM, Singapore (SG)

(21) Appl. No.: 18/034,115

(22) PCT Filed: Oct. 27, 2020
(86) PCT No.: PCT/SG2020/050616

§ 371 Date: Apr. 27, 2023

(51) Int. Cl. A61B 3/00; A61B 3/032; A61B 3/113

(52) U.S. Cl. CPC A61B 3/0041; A61B 3/032; A61B 3/113

(57) ABSTRACT

A system (200) for conducting a vision assessment test of an eye of a patient comprising: a display screen (201) for displaying at least one test profile to the patient; an eye tracker controlled for detecting a gaze direction data of the eye of the patient when viewing the test profile(s); and a processor for processing the detected gaze direction data and identifying a correlation between the gaze direction data and the test profile(s) to thereby obtain a vision assessment of the patients eye; wherein the system comprises a user interface (203) for enabling an operator of the system to author the test profile(s) for the patient by controlling one or more parameters of the test profile(s) prior to the vision assessment test being conducted.

### FIELD

The present invention generally relates to methods and systems for conducting a vison assessment test using authored test profiles. While the present invention will be described in particular for assessing ocular symptoms such as macular degeneration, it is to be appreciated that the invention is not restricted to this application, and that the testing of other ocular symptoms are also envisaged.

### BACKGROUND

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

Conventional test methods for assessing the macular degeneration of a patient, such as the use of an Amsler grid, preferential hyperacuity perimetry (PHP), and entoptic perimetry (EP), all have a number of disadvantages. These include the selected test not being the most effective for testing of a particular patient, the need for the patient to fixate on a target during each test which can lead to patient fatigue, and the requirement for oral reports from the patient which can be difficult for elderly patients. These tests also generally require a trained person to be present during that test to assess the collected test results.

U.S. Patent Publication No. 2019/0110678 (Agency for Science, Technology and Research and Tan Tock Seng Hospital), hereinafter referred to as the ‘U.S. patent publication’), describes an automated method and system for conducting a vision assessment test that addresses the above noted problems associated with conventional tests. The described method and system utilise a number of different predefined test patterns, and an automated method for assessing the vision functionality of the patient based on collected gaze data of that patient. The predefined test pattern is selected following a preliminary assessment of the eye of the patient to determine the best test pattern for assessing the vision acuity of that patient. The gaze of each eye of the patient is then tracked using an eye tracking device while the patient is tested using the selected test pattern. This automated method and system utilises a specific set of predefined test patterns, as described in paragraph of the U.S. patent publication. This can limit the efficacy of the test as the selected test pattern that may not always best meet the specific vison acuity test requirements of the patient being tested.

In a joint study, Augustinus Laude, Damon W K Wong, Ai Ping Yow, Muthu Mookiah, Tock H Lim; Eye gaze tracking and its relationship with visual acuity, central visual field and age-related macular degeneration features. Invest, Ophthalmol. Vis. Sci. 2018; 59(9):1264, involving the Applicants of the U.S. patent publication a correlation was shown between patients having age-related macular degeneration (AMD), and the eye movement performance (EMP) of that patient. The test involved patients following with their eye a computer-generated target moving in a sinusoidal waveform. Patients having AMD had greater difficulty in following the target, and therefore exhibited poorer EMP than patients not having this condition. The currently known test methods described in the U.S. patent publication do not however utilise this correlation in their test methods

An object of the invention is to ameliorate one or more of the above-mentioned difficulties.

### SUMMARY

According to an aspect of the present disclosure, there is provided a system for conducting a vision assessment test of an eye of a patient comprising:
a display screen for displaying at least one test profile to the patient;
an eye tracker controlled for detecting a gaze direction data of the eye of the patient when viewing the test profile(s);
and a processor for processing the detected gaze direction data and identifying a correlation between the gaze direction data and the test profile(s) to thereby obtain a vision assessment of the patient's eye;
wherein the system comprises a user interface for enabling an operator of the system to author the test profile(s) for the patient by controlling one or more parameters of the test profile(s) prior to the vision assessment test being conducted.

In some embodiments, the test profile comprises a test profile path and a pursuit target travelling along the test profile path, the vision assessment test requiring the patient's eye to following the pursuit target, and wherein the test profile path is determined by the operator of the system.

In some embodiments, the test profile path is determined by controlling the parameters comprising one or more of the following: waveform shape, waveform period, waveform frequency waveform amplitude, a-periodic or periodic waveform.

In some embodiments, the waveform shapes is selectable from sinusoid, square, rectangular, triangular, saw-toothed and 2^{nd} order waveform shapes.

In some embodiments, the test profile path comprises pulses and/or spikes displayed at a predetermined frequency or wavelength on the waveform.

In some embodiments, the test profile path comprises a geometric shape.

In some embodiments, the test profile path is selectable from an oval or rectilinear shape.

In some embodiments, the test profile path is an irregular curve in shape.

In some embodiments, the authored test profile path is a wide pursuit path that extends across the entire threshold of the screen or field-of-vision of the patient.

In some embodiments, said test profile comprises a plurality of target images being separately displayed on the display screen in a pseudo-random pattern during the vision assessment test.

In some embodiments, the user interface enables the operator of the system to author the test profile by controlling parameters comprising one or more of the following: number of the test targets to be displayed, size of the test target, colour of the test target, colour of the background for the test.

In some embodiments, the user interface further enables a sensitivity of the eye gaze detection being used for the test, the sensitivity being indicated on the user interface by a boundary ring encircling each of the target images.

In some embodiments, the user interface is displayed on the display screen.

According to another aspect of the present disclosure, there is provided a method for conducting a vision assessment test of an eye of a patient comprising:
conducting a preliminary vision assessment of the patient;
authoring at least one test profile by controlling one or more parameters of the test pattern on a user interface taking into account the preliminary vision assessment;
displaying the one or more test profiles on a display screen to the patient;
collecting data on the patient's gaze direction in response to the one or more test patterns displayed to the patient; and
conducting a vision assessment of the patient based on identifying a correlation between the gaze direction data and the one or more test patterns.

Other aspects and features will become apparent to those of ordinary skill in the art upon review of the following description of specific embodiments in conjunction with the accompanying figures.

### BRIEF DESCRIPTION OF THE DRAWINGS

In the figures, which illustrate, by way of example only, embodiments of the present disclosure,

FIG. 1 is a flow chart of the prior art automated system and method for vision assessment of a patient as described in the U.S. patent publication;

FIG. 2 shows a display screen and an associated UI software interface for a system for conducting a vision assessment test of an eye of a patient using an authored test profile path according to an aspect of the present disclosure;

FIGS. 3A and 3B respectively show test profile paths generated using second order ordinary differential equations (ODE), and test profile paths generated using a Fourier equation that can be inputted into the system and method according to the present disclosure;

FIGS. 4A and 4B respectively show an oval and rectangular test profile path that can be authored for the system and method according to the present disclosure;

FIG. 5 is a flow chart of the operation of the system for conducting a vison assessment test according to the present disclosure;

FIG. 6 is a flow chart showing details of the pseudo-random generator finite state used for the system of FIG. 5;

FIGS. 7 and 8 are images respectively of the UI software interface provided on the display screen of the system and method according to the present disclosure;

FIGS. 9 to 12 are images of the pseudo-random pattern of test targets (only one of which is displayed at any one time on the display screen) generated by the system and method according to an aspect of the present disclosure; and

FIGS. 13 to 16 are images of the authored test profiles and test results displayed on the display screen according to an aspect of the system and method of the present disclosure.

### DETAILED DESCRIPTION

Throughout this document, unless otherwise indicated to the contrary, the terms “comprising”, “consisting of”, “having” and the like, are to be construed as non-exhaustive, or in other words, as meaning “including, but not limited to”.

Furthermore, throughout the specification, unless the context requires otherwise, the word “include” or variations such as “includes” or “including” will be understood to imply the inclusion of a stated integer or group of integers but not the exclusion of any other integer or group of integers.

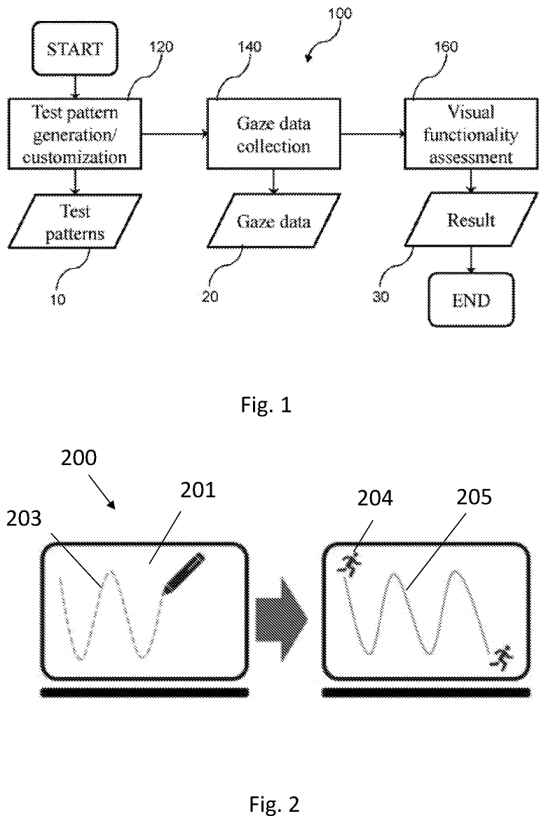

The present disclosure is directed to an improvement in the system and method described in the above-mentioned U.S. Patent Publication, details of which are incorporated herein by reference. FIG. 1 is a flowchart 100 of the automated method for vision assessment from the U.S. patent publication. The method initially comprises conducting a preliminary vison assessment of the patient and selecting suitable test patterns for testing the patient based on that preliminary vision assessment. This preliminary assessment may include imaging of a fundus of the eye to detect damage and/or anomalies in the eye. This is shown in the flow chart 100 as the ‘Test pattern generation/customization’ step 120 from which can be selected an appropriate test pattern(s) 10 for the patient. The test can then be conducted on the patient using a number of test patterns 10, with the gaze of the patient being automatically traced and recorded using an eye tracker to enable the gaze direction data to be recorded. The U.S. patent publication refers to a commercially available eye tracking machine that can be used in their system. The gaze direction data will help to identify the degree of correlation of the patient's gaze with the test patterns being displayed. This is shown as the ‘Gaze data collection’ step 140 in the flow chart 100, from which is collected gaze data 20 for that patient. A visual functionality assessment of the patient can then be conducted based on this collected gaze data 20. This is referred to as the “Visual functionality assessment’ step 160 from which can be obtained the test results 30 for that patient.

The test system and method according to the U.S. patent publication utilizes a series of predefined test patterns from which the most appropriate test patterns 20 can be selected. It is however to be appreciated that the use of predefined test patterns can be limiting as it may not always be possible to ensure that the patient can be tested using test patterns most appropriate to their specific vision condition. Furthermore, the use of predefined test patterns does not allow for any changes to be made to the test patterns during or immediately prior to the commencement of the vision tests.

The system and method for conducting a vision assessment test according to the present disclosure also requires that a preliminary assessment be made of the patient's eye prior to conducting of the eye test. FIG. 2 shows an aspect of the system and method according to the present disclosure. Rather than using predefined test patterns as described in the U.S. patent publication, the clinician operating the system according to the present disclosure can author a test profile that is applicable for testing the eyes of the patient. One aspect of the system and method of the present disclosure provides means to allow for a test profile based on a pursuit image 204 following a test profile path 205 to be authored by the clinician at the time of testing of a patient to take into account the specific vison condition of the patient. The system 200 includes a display screen 201 as shown in FIG. 2 for displaying the pursuit image 204 that travels along a test profile path 205 generated by the system 200. The display screen 201 provides an UI software interface 203 that facilitates the generation of a test target presentation on a test profile path 205 authored by the operator of the system The authored test profile path may be a wide pursuit test profile path that requires the gaze of the patient to move across the entire threshold of the screen or field-of-vision (POV). The UI software interface 203 presents options to the operator to for example generate pursuit target waveform types and shapes. It is also envisaged that the UI Software interface may be shown on a separate screen other than on the display screen 201.

The path-waveform generator can be set to generate an output target path on a sinusoid or other shapes of signal waveform such as square Waves, rectangular Waves, Triangular Waves, saw-toothed waveform and a variety of pulses and spikes at some predetermined frequency or wavelength that can be generated along a said waveform The tester can configure and preset visual representations of a linear a-periodic or periodic waveform following three common characteristics:
Period: This is the length of time in seconds that the waveform takes to repeat itself from start to finish. This value can also be called the Periodic Time, (T) of the waveform for sine waves, or the Pulse Width for square waves.
Frequency: This is the number of times the waveform repeats itself within a one second time period. Frequency is the reciprocal of the time period, (f=1/T) with the standard unit of frequency being the Hertz, (Hz).
Amplitude: This is the magnitude or intensity of the signal which is the Y-axis displacement across the screen.

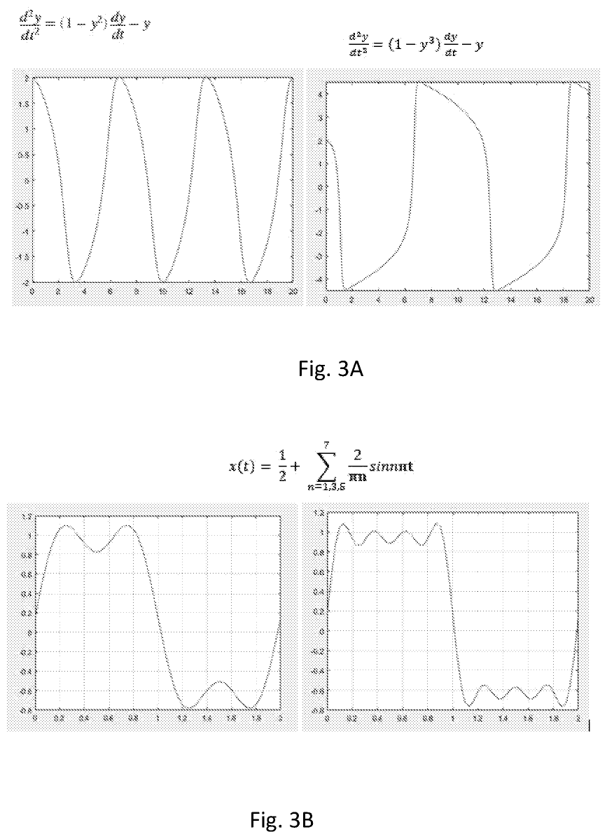

The above described three characteristics may be adjustable settings within the system and method according to the present disclosure. It is however to be appreciated that the test profiles are not limited to first order waveforms. For example, a second order waveform could be generated by inputting a Fourier equation as shown in FIGS. 3A and 3B. FIG. 3A shows an example of a second order ordinary differential equation (ODE) and resultant test profile curve that can be inputted into the system and method according to the present disclosure. FIG. 3B is another example of a Fourier equation and resultant test profile curve that can be inputted into the system and method according to the present disclosure.

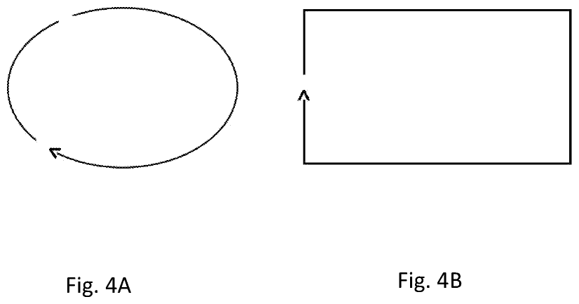

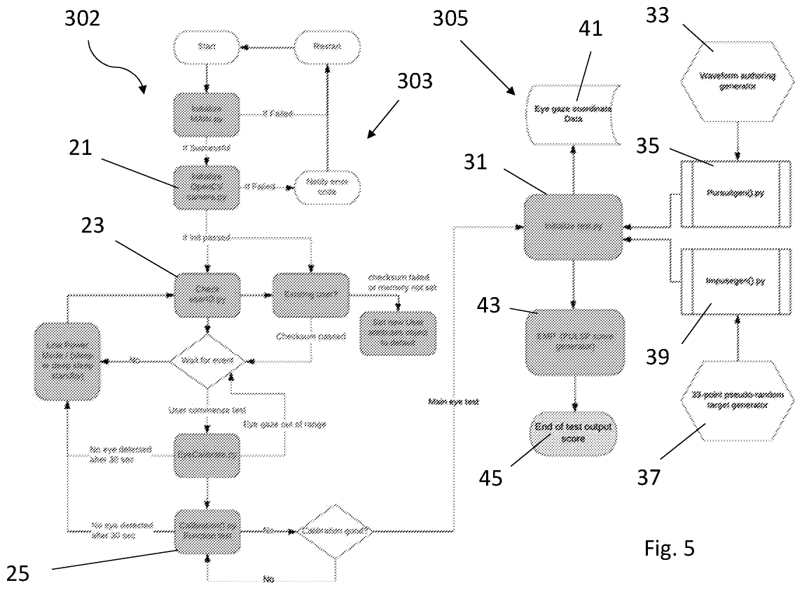

The system and method according to the present disclosure may also not be limited to the use of authored test profiles in the form of waveforms. It is also envisaged that other patterns such as geometric shapes could be used. For example, the operator of the system can an oval extending across the screen as shown in FIG. 4A. This then allows for the gaze of the patient to trace from left-to-right of the screen and then right-to-left of the screen. Alternatively, the operator can draw a rectilinear shape such as a rectangle, where the rectangular test path reverses across the screen as shown in FIG. 4B.

A pursuit image 204 can then be displayed on the display screen 201 of the electronic apparatus 200 following the path of the configured test profile path 205. The gaze direction of the patient's eyeball is controlled by six eye muscles pulling and pushing the eyeball in different directions. The performance of these eye muscles in controlling the gaze direction can be tested by having the patient's gaze follow the generated pursuit image 204, following different test profile paths 205. The patient's gaze direction is detected to determine how closely the gaze direction follows the pursuit image 204. As discussed in the previously referred to study, a poor following of the pursuit image by the patient's eye can indicate that the patient is suffering AMD. The use of different test profile shapes facilitates the testing of the eye muscles that move the eyeball in specific movement orientations. For example, a square/rectangular waveform can allow testing of the eye muscles alternatively moving the gaze in vertical and horizontal directions. Alternatively, the test profile path may be a geometric shape such as an oval or rectangle as previously described. The pursuit target 204 can also be made to generally move from left to right of the screen, or alternatively from the right to left of the screen if preferred.

According to another aspect of the present disclosure, a series of pseudo-random target images may be displayed on the screen 201 which seeks to address a ‘learned response’ issue. Each target image may be separately displayed on the display screen 201 in front of the patient being tested. Each target image can be separately displayed as a spot on the screen 201, which each target image being displayed in a pseudo-random pattern. It is to be appreciated that the use of other target shapes other than a spot is also envisaged. The advantage in using a pseudo-random pattern is that it addresses the situation where the patient has learned to predict when the next target image will appear. This can arise when the patient has previously been tested using such a test pattern and has learnt when the next target image is likely to arise thereby negating the efficacy of that test. This will affect the collected gaze data of that patient if they are able to predict where the next target image is likely to be displayed. Having the target image being generated to a location within a test pattern in a pseudo-random order therefore prevents the test results being influenced due to prior learning of the expected display locations of each target image when displayed.

FIG. 5 is a flow chart 302 showing the steps for the overall operation of the system 200 when used according to the present disclosure. The flow cart 302 is separated into the system setup steps 303 shown on the left side of the flow chart 302, and the test steps 305 on the right side of the flow chart 302 according to the present disclosure. The system setup steps 303 includes initialisation steps 21 of the system and eye tracker camera, user ID verification steps 23, and calibration steps for ensuring that the eye of the patient is correctly positioned for gaze tracking by the eye tracker. These steps are conducted prior to the main eye test being conducted as shown in the test steps 305.

Once the patient is correctly positioned to enable their eye gaze coordinates to be recorded, the main eye test can be initiated (43). The test to be conducted can then be selected from the two tests as previously described. If the test shown in FIG. 2 is selected, then a waveform authoring generator (33) can be used by the clinician to generate a sinusoid, square, rectangular, triangular, sawtooth or other waveform having a period, frequency and/or amplitude selected based on the vison assessment requirements of the patient using a software algorithm (35) run within a processor of the system. Alternatively, a geometric test profile path could be authored as previously described. The patient can then be asked to follow a pursuit target moving along the generated test profile path with the eye that is being tested. Alternatively, the patient can be asked to follow with their eye target images as they are generated in a pseudo-random manner by a software algorithm (39) run within the system processor. The eye gaze coordinates of that eye can be collected as data (41). That data can be used to generate a test score (43) to thereby provide a final test output score for the patient (45).

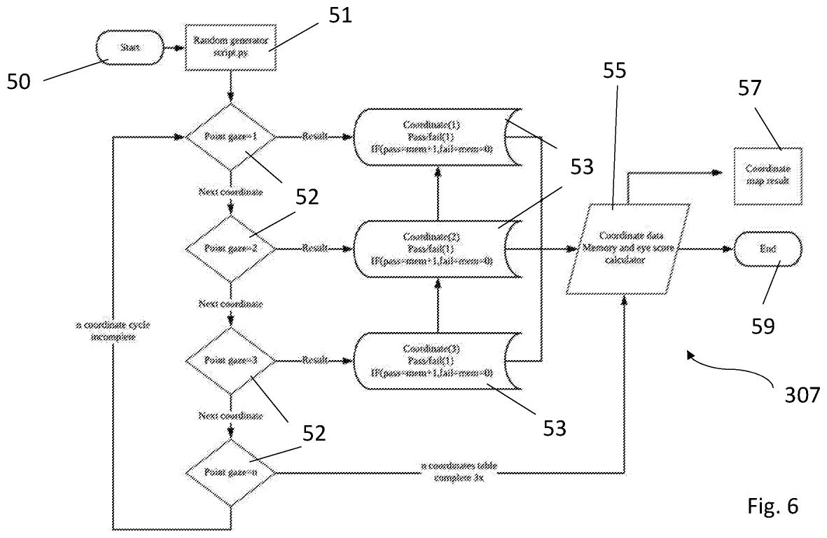

FIG. 6 is a flow chart 307 which shows in more detail the operation of the eye score generator algorithm when used in relation to the pseudo-random target generator. Following the start of the test procedure (50), a test spot coordinate (52) is generated by the random generator (51). At each point gaze coordinate (52), a score is determined based on whether or not the patient has appropriately moved their gaze to the generated coordinate (53). A score of 1 is recorded if the patient has passed the gaze test, while a score of 0 is recorded if they fail the gaze test at that coordinate. These scores are added to calculate a final eye score (55), and a coordinate map is prepared (57), or the test is ended (59).

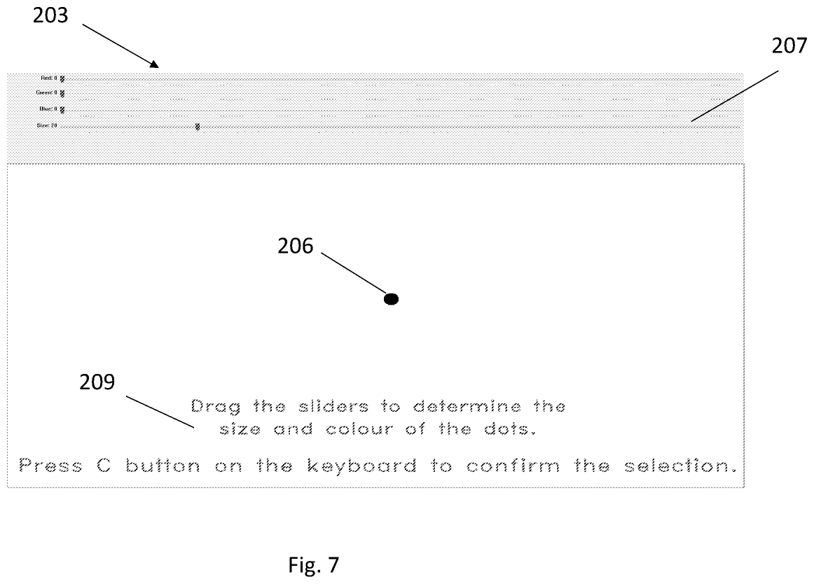

FIGS. 7 to 16 show in more detail the images provided by the UI software interface 203 of the system 200 and the test targets and test profiles and results provided on the display screen 201 of the system according to the present disclosure.

FIG. 7 is an image of the UI software interface 203, which may be shown on the display screen 201, for the authoring function of the creation of the target image 206 shown as a dot in the middle of the interface 203. This target image 206 can be used as part of the pseudo-random target image test. Software sliders 207 are provided on the top of the interface 203 for adjusting the colour in RGB hue and the size of the target image 206 in pixels. Instructions 209 are provided at the bottom of the interface 203 for the operator of the system.

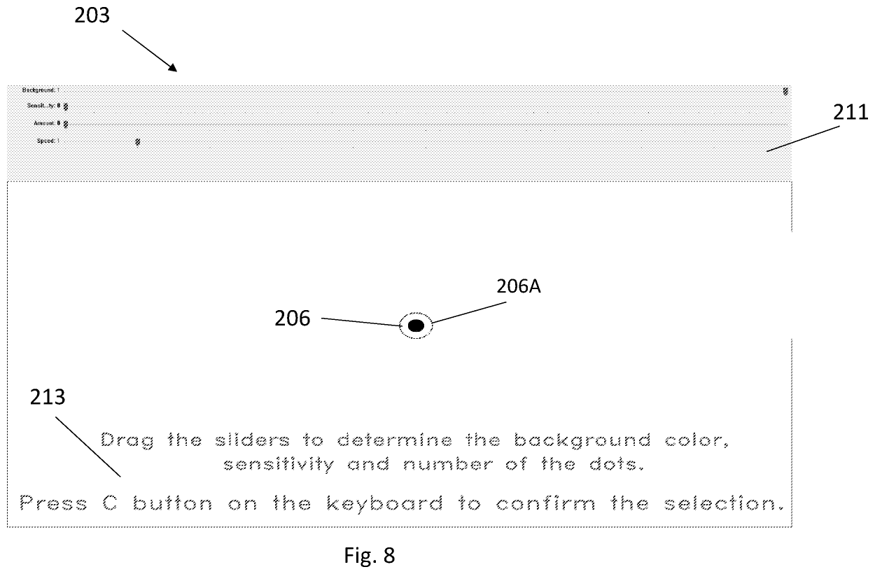

FIG. 8 is an image of another UI software interface 203 showing the adjustment function for the target images 206. These adjustments may be controlled by software sliders 211 provided at the top of the interface 203. The adjustments can include the number of target images 206 to be shown during the pseudo-random target image test, the selection of the background colour which may for example be changed between white or black, the sensitivity (or boundary conditions where the target image will be registered as gaze-positive) of the gaze data detection step of the system shown as boundary ring 206A surrounding the target image 206, and the speed of display of the target images 206. Instructions 213 are also provided at the bottom of the interface 203 for the operator of the system.

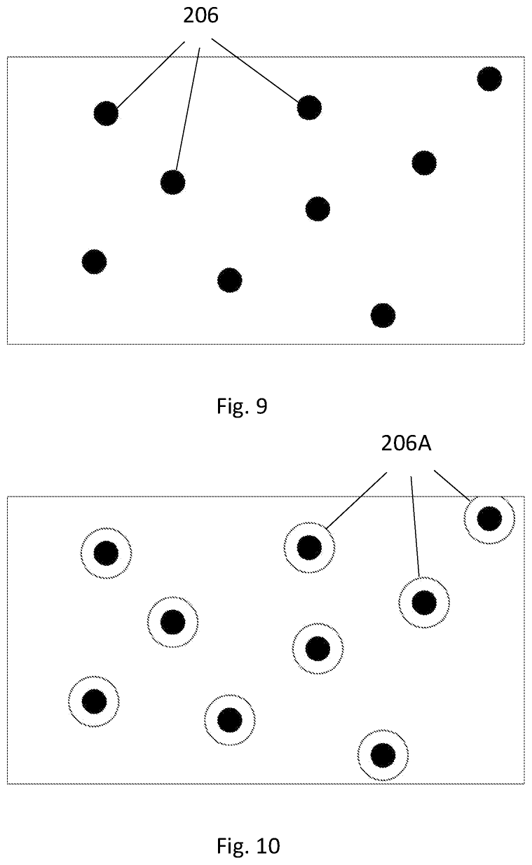

FIG. 9 is an image of nine target images 206 authored and adjusted using the previously described UI software interface 203, and to be used for the pseudo-random target image test. The target images may be displayed to the operator prior to the pseudo-random test being conducted on a patient. Each of these target images 206 will however be separately displayed in a pseudo-random position during the test, and that the patient will therefore only be displayed a single target image 206 at any point in the test, and may not view all of the target images 206 at one time as shown in FIG. 9.

FIG. 10 is an image of the same nine target images 206 shown in FIG. 9, but with the boundary ring 206a representing the boundary conditions surrounding each target image 206 being visible. As noted previously, this boundary ring 206A represents the sensitivity of the eye gaze detection being used for the test. These boundary circles 206A represent boundary regions that should not be overlapping, and so the boundary ring 206A indicates the minimum distance that the target images 206 will be separated from each other during the test.

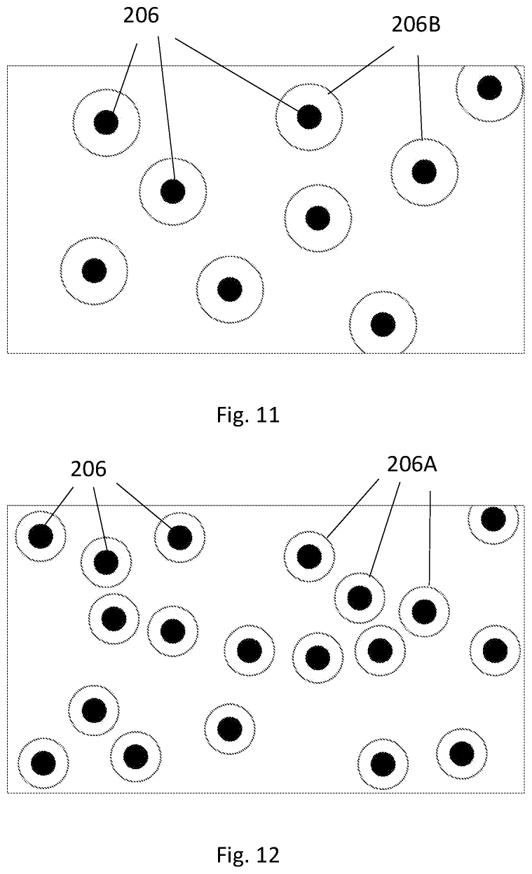

FIG. 11 is an image of the same nine target images 206 shown in FIG. 9, but with boundary rings 206B representing a wider boundary region surrounding each target image 206 being visible. This indicates that a lower sensitivity is being used for the eye gaze detection during the test. In other words, the patient being tested is being provided with a greater leeway in the detection of a gaze positive by the eye gaze detection of the system 200.

FIG. 12 is an image of a greater density of target images 206 (nineteen are shown in FIG. 12) authored and adjusted using the UI software interfaces 202,203 shown in FIGS. 7 and 8. Boundary rings 206A are displayed around each target image 206 denoting a higher sensitivity than as shown in FIG. 11. The boundary regions denoted by each boundary ring 206A must also not overlap, and the boundary rings 206A therefore indicate the minimum distance that the target images 206 can be separated from each other.

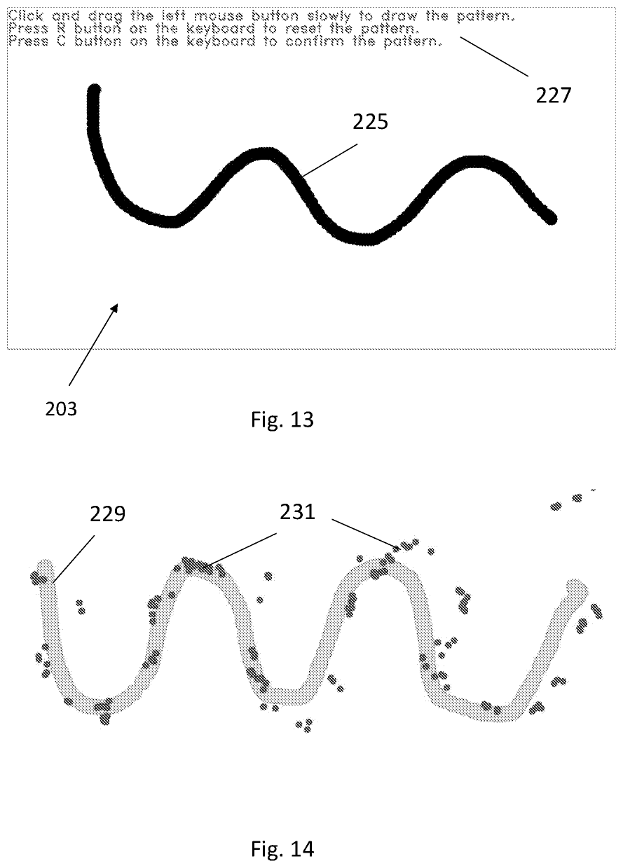

FIG. 13 is an image of a UI software interface 203 on the display screen 201 that allows a test profile path 225 to be authored on the interface 203, for example by using a computer mouse. The top of the interface 203 is provided with instructions 227 for the operator of the system instructing them on how to author the test profile path 225 to which a pursuit target (not shown) will travel during the test where the patient's gaze is tracked while following the pursuit target.

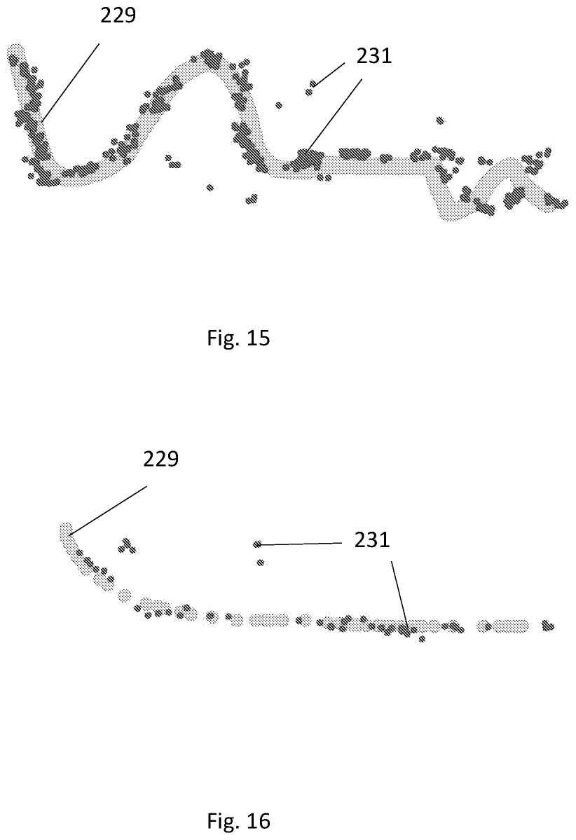

FIGS. 14 to 16 respectively show the results obtained from such a test, with the green line representing different test profile paths 229 authored by the operator for different or the same patient being tested. The test profile path 229 may also be an irregular curve in shape as shown in FIGS. 14 to 16 and may not therefore be restricted to waveform shaped or geometric shaped paths as previously described. The red dots 231 represent the actual position of the patient's gaze measured while following the pursuit target travelling along the test profile path 229. The test result shown in FIG. 13 show the patient having a lower ability to maintain their gaze on the pursuit target than the results shown in FIGS. 15 and 16 thereby indicating possible macular degeneration in the patient.

The facility to allow for test profiles to be authored by the operator of the system according to the present disclosure is advantageous because it allows the test profiles to be specifically authored to take into account the specific vision condition of the patient. This may then help to ensure that the vison assessment test conducted using the system and method according to the present disclosure can provide a more precise test result for that patient.

Throughout the description, it is to be appreciated that the term ‘processor’ and its plural form include microcontrollers, microprocessors, programmable integrated circuit chips such as application specific integrated circuit chip (ASIC), computer servers, electronic devices, and/or combination thereof capable of processing one or more input electronic signals to produce one or more output electronic signals. The processor includes one or more input modules and one or more output modules for processing of electronic signals.

Unless defined otherwise, all technical and scientific terms used herein have the same meaning as is commonly understood by a skilled person to which the subject matter herein belongs.

It should be appreciated by the person skilled in the art that the above invention is not limited to the embodiment described. It is appreciable that modifications and improvements may be made without departing from the scope of the present invention.

It should be further appreciated by the person skilled in the art that one or more of the above modifications or improvements, not being mutually exclusive, may be further combined to form yet further embodiments of the present invention.

1. A system for conducting a vision assessment test of an eye of a patient comprising:
a display screen for displaying at least one moving target test profile to the patient;
an eye tracker controlled for automatically detecting a gaze direction data of the eye of the patient when tracking the test profile(s);
and a processor for processing the detected gaze direction data and identifying a correlation between the gaze direction data and the test profile(s) to thereby obtain a vision assessment of the patient's eye;
wherein the system comprises a user interface for enabling an operator of the system to author the test profile(s) for the patient by controlling one or more parameters of the test profile(s) prior to the vision assessment test being conducted.

2. A system according to claim 1, wherein the test profile comprises a test profile path and a pursuit target travelling along the test profile path, the vision assessment test requiring the patient's eye to following the pursuit target, and wherein the test profile path is determined by the operator of the system.

3. A system according to claim 2, wherein the test profile path is determined by controlling the parameters comprising one or more of the following: waveform shape, waveform period, waveform frequency waveform amplitude, a-periodic or periodic waveform.

4. A system according to claim 3, wherein the waveform shapes is selectable from sinusoid, square, rectangular, triangular, saw-toothed and 2^{nd} order waveform shapes.

5. A system according to claim 2, wherein the test profile path comprises pulses and/or spikes displayed at a predetermined frequency or wavelength on the waveform.

6. A system according to claim 2, wherein the test profile path comprises a geometric shape.

7. A system according to claim 6, wherein the test profile path is selectable from an oval or rectilinear shape.

8. A system according to claim 2, wherein the test profile path is an irregular curve in shape.

9. A system according to claim 2, wherein the authored test profile path is a wide pursuit path that extends across the entire threshold of the screen or field-of-vision of the patient.

10. A system according to claim 1, wherein said test profile comprises a plurality of target images being separately displayed on the display screen in a pseudo-random pattern during the vision assessment test.

11. A system according to claim 10, wherein the user interface enables the operator of the system to author the test profile by controlling parameters comprising one or more of the following: number of the test targets to be displayed, size of the test target, colour of the test target, colour of the background for the test.

12. A system according to claim 11, wherein the user interface further enables a sensitivity of the eye gaze detection being used for the test, the sensitivity being indicated on the user interface by a boundary ring encircling each of the target images.

13. A system according to claim 1, wherein the user interface is displayed on the display screen.

14. A method for conducting a vision assessment test of an eye of a patient comprising:
conducting a preliminary vision assessment of the patient;
authoring at least one moving target test profile by controlling one or more parameters of the test profile on a user interface taking into account the preliminary vision assessment;
displaying the one or more test profiles on a display screen to the patient;
automatically collecting data on the patient's gaze direction in response to the eye of the patient tracking the one or more test profiles displayed to the patient; and
conducting a vision assessment of the patient based on identifying a correlation between the gaze direction data and the one or more test profiles.

15. A method according to claim 14, wherein the test profile comprises a test profile path and a pursuit target travelling along the test profile path, the vision assessment test requiring the patient's eye to following the pursuit target, and wherein the test profile path is determined by the operator of the system.

16. A method according to claim 15, wherein the test profile path is determined by controlling the parameters comprising one or more of the following: waveform shape, waveform period, waveform frequency waveform amplitude, a-periodic or periodic waveform.

17. A method according to claim 16, wherein the waveform shapes is selectable from sinusoid, square, rectangular, triangular, saw-toothed, and 2^{nd} order waveform shapes.

18. A method according to claim 15, wherein the test profile path comprises pulses and/or spikes displayed at a predetermined frequency or wavelength of the waveform.

19. A method according to claim 15, wherein the test profile path comprises a geometric shape.

20. A method according to claim 19, wherein the test profile path is selectable from an oval or rectilinear shape.

21. A method according to claim 15, wherein the test profile path is an irregular curve in shape.

22. A method according to claim 15, wherein the authored test profile path is a wide pursuit path that extends across the entire threshold of the screen or field-of-vision of the patient.

23. A method according to claim 14, comprising using said test profile having a plurality of target images separately displayed on the display screen in a pseudo-random pattern during the vision assessment test.

24. A method according to claim 23, wherein the user interface enables the operator of the system to author the test profile by controlling parameters comprising one or more of the following: number of the test targets to be displayed, size of the test target, colour of the test target, colour of the background for the test.

25. A method according to claim 24, wherein the user interface further enables a sensitivity of the eye gaze detection being used for the test, the sensitivity being indicated on the user interface by a boundary ring encircling each of the target images.

26. A method according to claim 25, wherein the user interface further enables a sensitivity of the eye gaze detection being used for the test, the sensitivity being shown on the user interface by a boundary region encircling each of the target images.

27. A method according to claim 15, wherein the user interface is displayed on the display screen.
